(19) United States
(12) Patent Application Publication
(10) Pub. No.: US 2026/0090863 A1
(43) Pub. Date: Apr. 2, 2026

(54) ELECTROSURGICAL INSTRUMENT HAVING TRANSPONDER, TRANSPONDER COMMUNICATIONS SYSTEM AND PRODUCTION METHOD

(71) Applicant: Aesculap AG, Tuttlingen (DE)

(72) Inventors: Ralf Pfister, Trossingen (DE); Frederick Lenzenhuber, Tuttlingen (DE); Maximilian Bieber, Filderstadt (DE)

(21) Appl. No.: 19/114,093

(22) PCT Filed: Sep. 14, 2023
(86) PCT No.: PCT/EP2023/075270

§ 371 Date: Mar. 21, 2025

(30) Foreign Application Priority Data
Sep. 23, 2022 (DE) 10 2022 124 571.3

(51) Int. Cl. A61B 90/98; A61B 17/00; A61B 18/00; A61B 18/14; G06K 7/10

(52) U.S. Cl. CPC A61B 90/98; A61B 18/1442; G06K 7/10366; A61B 2017/00526; A61B 2018/00083; A61B 2018/1462

(57) ABSTRACT

An electrosurgical instrument, in particular an RF instrument, includes an electrode device with a first electrode and a second electrode that are opposite one another. An insulation body accommodates a transponder, preferably an RFID transponder, particularly preferably a glass transponder. The insulation body connects the first electrode and the second electrode to one other. The transponder is arranged between the first electrode and the second electrode, in particular symmetrically and/or centrally between the first and the second electrodes. A screen is formed in the first electrode and/or in the second electrode in the region of the transponder such that the first electrode and/or second electrode forms a stop that does not allow the passage of electromagnetic wave signals. A screen opening allows passage of the signals. The electrosurgical instrument can be part of a medical transponder communications system and manufactured by a production method.

### CROSS-REFERENCE TO RELATED APPLICATIONS

This application is the United States national stage entry of International Application No. PCT/EP2023/075270, filed on Sep. 14, 2023, and claims priority to German Application No. 10 2022 124 571.3, filed on Sep. 23, 2022. The contents of International Application No. PCT/EP2023/075270 and German Application No. 10 2022 124 571.3 are incorporated by reference herein in their entireties.

### FIELD

The present disclosure relates to a medical electrosurgical instrument, in particular an HF instrument, particularly preferably a bipolar HF instrument, with a transponder adapted to receive and/or transmit electromagnetic waves, in particular data signals. The invention furthermore relates to a medical transponder communication /stem/ transponder system for communicating with and reading and/or writing to a transponder as well as to a production method, in particular an assembly method.

### BACKGROUND

Assemblies with transponders or (medical) marking elements with RFID transponders or RFID tags are known from the state of the art, which are used in particular for equipping surgical instruments. With the help of the RFID transponders attached to the surgical instruments, it is possible to identify, track and manage such an instrument. In particular, specific information about the instrument can be read out.

For example, EP 3 193 284 A1 discloses a medical marking element for equipping surgical instruments, which can be subsequently attached to the surface of a surgical instrument. The marking element has a ring-shaped metal frame with a non-conductive cover, in the interior of which an RFID transponder/RFID tag is inserted. An outer side of the metal frame is attached to the surgical instrument in a predetermined position, in particular by welding.

In particular in the field of electrosurgical high-frequency (HF) instruments, such as bipolar HF instruments, these instruments have a limited service life in terms of application cycles as well as processing cycles in which the instrument is deployable and usable. These cycles have to be strictly adhered to. This is where a transponder is fit for the task. The transponder can be used to clearly identify the electrosurgical instrument and thus also to detect the associated application cycles and processing cycles. In this way, the instrument can be detected, logged and tracked in order to indicate a necessary replacement or service of the individual instrument. For example, a counter associated with an instrument ID can be incremented and, if necessary, reset to zero after a replacement or service.

However, a disadvantage of the prior art is that transponders, in particular passive transponders, have to be arranged at a short distance from a reading device or reading and writing device due to their short (signaling) transmission range. This applies in particular to NFC (Near Field Communication) RFID tags. For this reason, with conventional transponders/RFID tags it is often only possible to subsequently apply the RFID tags to an exposed and easily accessible outer surface of a medical instrument in order to minimize the distance between the transponder and the reading device. Care has to be taken to ensure that the outer surface provided for this purpose is located at an instrument site that has only minor disadvantages in terms of instrument handling and at the same time ensures sufficient transmission quality and sufficient reception. It is also important to separate the RFID tag from the current-carrying and voltage-carrying components in the region of the electrosurgical instruments.

In addition, transmission, reception and transmission power are significantly influenced by the components and structures surrounding the transponder. This also affects the (signal-related) reliability of transponder transmission. In particular, however, the maximum possible distance to a reading and/or writing device changes so that, depending on the structure of the instrument and a location where an RFID marking element is attached, reading is no longer possible or at least not possible with sufficient reliability. The geometric mounting situation of an RFID marking element and thus of an RFID chip of the transponder has an essential effect on this maximum distance and varies from instrument to instrument, so that safe, reliable and predictable handling is not possible with the marking elements according to the state of the art.

Therefore, when developing a design for a medical instrument, an RFID marking element and an environment of an RFID tag always has to be taken into account and incorporated into the design, since this is the only way to ultimately ensure that a suitable surrounding structure is available for the transponder. This makes it difficult to develop a medical instrument independently of the transponder. Since the approval of medical products is lengthy and very expensive, both existing and new medical products face a particularly high hurdle with the corresponding challenge of adaptation.

In addition to the signal-related problems, the state of the art also has the following disadvantages. Although such RFID marking elements are comparatively small components, the marking elements with RFID transponders subsequently applied in the prior art form additional contact surfaces for contamination and are also a hindrance when handling the medical instruments. Additional RFID marking elements attached to the surface may also form gaps and cracks in which germs can accumulate. Also, due to the design of the marking elements and their attachment to a medical instrument, irreversible attachment, in particular welding, is unavoidable and makes it difficult to replace the transponder in the event of a defect or an intended change. Furthermore, due to their sharp edges, externally attached marking elements always pose a risk of tearing open a surgical glove and harming the patient and user.

The manufacture and assembly of a marking element, in particular via welding, is also very complex and in some cases not possible at all for some medical instruments, since no suitable materials are available on a surface for welding or as provided mounting surfaces are not suitable. In particular, the parts of a housing or holder of the RFID marking element always have to be made of metal in order to be attached and welded on, which limits the choice of suitable materials for a design. In addition, there are specifications from the approval of medical instruments, which in particular require the RFID marking element to be manufactured and mounted with repeatable accuracy. Metallic surfaces also shield RFID-marking elements and reduce their accessibility.

### SUMMARY

It is therefore the object of the present invention to eliminate or at least reduce the disadvantages of the prior art and in particular to provide an electrode device, a medical instrument with a transponder, a transponder communication system as well as a manufacturing process which ensures the reliability of signal-related communication and improves the reception or reception power and/or transmission power of a transponder, in particular with regard to a distance to a reading and writing device. In addition, cleanability, sterilizability as well as good and safe handling of a medical instrument and of a transponder are to be improved. The development, manufacture, assembly, maintenance, repair as well as replacement of an electrosurgical instrument are also to be improved. The transponder can be changed quickly and easily during servicing.

In other words, it is in particular the object of the invention to provide a medical electrosurgical instrument, in particular an HF instrument, which ensures good and safe handling both with regard to the instrument property and with regard to the data transmission property of the transponder. A further object is preferably good cleanability and/or sterilizability of the medical electrosurgical instrument.

The object is solved with regard to a generic (medical) electrosurgical instrument according to the invention and with regard to a generic medical transponder communication system according to the invention and with regard to a production method according to the invention.

A basic idea of the present disclosure can therefore be seen in the fact that in an electrosurgical instrument with at least two electrodes, a further, non-conductive insulation body (i.e. with a very high resistance) is provided, which houses a transponder and is connected to the electrodes in such a way that the transponder is arranged between the electrodes. This means that the transponder is spaced apart from the electrodes via the insulation body and is therefore electrically insulated, and the transponder is also set back from the electrodes toward an environment. In at least one electrode of the two electrodes, in particular in both electrodes, a passage opening is integrated in the region of the transponder, so that the passage opening creates a screen opening to the environment. This favors a read and write capability of the transponder, which can be read in this way not only in a top view of the two electrodes, but also from a direction in which the electrode is arranged between the transponder and a transponder reader.

At in particular one proximal region of the at least two electrodes, a transponder mounted in an insulation body is thus integrated between the electrodes and, in addition, data transmission (readout capability) is significantly improved by the screen opening in at least one electrode.

In other words, a receptacle of a transponder, in particular of an RFID tag, especially particularly of a glass tag in HF instruments is proposed in a proximal insulation body between the two opposing electrodes, preferably in the center of the outer contour of the insulation body.

Through corresponding geometric recesses/openings in at least one electrode, in particular both, of the opposing electrodes in the region of the transponder (in particular of the glass tag), the transponder can be read out in all directions or through 360°.

In yet other words, an electrosurgical instrument, in particular an HF instrument, (with an electrode device) is provided with a first electrode and a second electrode which lie opposite each other, in particular distally of the instrument. Furthermore, the instrument has an (electrical) insulation body/an insulation module which is configured in an electrically insulating manner, in which a transponder, preferably an RFID transponder, particularly preferably a glass transponder, is housed/inserted. This insulation body connects the first and second electrodes to each other, wherein the transponder housed in the insulation body is arranged between the first and second electrodes, in particular symmetrically and/or centrally between the first and second electrodes. Furthermore, a geometric recess/opening/screen opening is configured in the first electrode and/or in the second electrode in the region of the transponder or at the level of the transponder, so that the electrode forms a screen impermeable to signals of electromagnetic waves with a screen opening permeable to signals.

The transponder is housed in the insulation body (electrically insulating body) and is fixed in position. So while the electrode is configured from metal, for example, in order to conduct the electric current, this material also means that the electrode is impermeable to signals. By creating a special geometric recess or screen opening in the electrode, signal-related transmission can also be provided in the direction of the electrode, i.e. in an extension of a straight line from the transponder to the electrode, toward an environment. If such a screen
opening is now provided in the at least two electrodes, in particular in all electrodes, signal-related data transmission is possible in 360° around the transponder. In particular, the screen opening even increases the maximum possible distance to a reading device. This means that a screen with predefined geometric dimensions (such as the screen opening) is configured directly in the electrode, which improves the reception of the transponder and, in particular, the possible distance to an external reading and/or writing device.

The transponder can thus be integrated even better and more flexibly into the electrosurgical instrument.

The insulation body itself is in particular signal-permeable and preferably has no or only little influence on a (signal-related) reception of the transponder or on an electromagnetic interaction. The insulation body may be configured in particular with regard to good cleanability and sterilizability and also with regard to installation or insertion in the medical instrument or the electrodes in the insulation body.

The screen or the electrode with the screen opening, which has a material impermeable to signals or a material that is not permeable to electromagnetic waves, at least for a certain frequency range, or consists of such a material, behaves completely differently. The screen opening has an essential influence on an electromagnetic interaction between the transponder and an environment and is in particular specially configured and adapted to bundle and/or amplify electromagnetic signals or electromagnetic waves of the transponder.

Due to this configuration, the electrode device of the electrosurgical instrument is optimally configured as a unit, and reception and transmission to and from the transponder to the environment is significantly improved. As a result, communication between a (transponder) reading and/or writing device that may be arranged in the region of the upper side or the surface of the instrument body is also improved, so that secure and reliable reading and/or writing of the transponder is ensured by the configuration of the electrode device itself, even over longer distances.

The term ‘in the region of the transponder’ means, for example, that the screen opening is provided at a similar height along a longitudinal axis of the instrument, in particular in a region of the electrode that is geometrically closest to the transponder.

It is emphasized at this point that the electrode device with the first and second electrodes and the insulation body with the transponder constitutes an independent invention.

The instrument with transponder may be used, for example, to detect and document a product-related reprocessing cycle with corresponding information and to further process the knowledge gained from it. Such a reprocessing cycle may, for example, include cleaning and/or sterilization and/or oiling. The instrument with transponder may be used for tracking and lifecycle management of a medical device/product, in particular of a medical instrument. The information also serves the manufacturer with regard to evidence in the event of a complaint. Furthermore, a user may only subject a medical device fitted with the transponder, in particular a medical instrument, to maintenance when maintenance is individually required and no longer has to adhere to a predefined maintenance interval. This favors the availability and provision of medical instruments, since the maintenance intervals may be individually extended.

Advantageous embodiments are explained in particular below.

According to one embodiment, the screen opening of the electrode may be elongated or slit-shaped or slot-shaped, in particular may be configured as a slot hole. In particular in the case of electrodes which extend in one direction like tweezers and have a sheet-like or plate-like basic structure, a screen opening may be integrated into the electrode itself by inserting an elongated recess.

In one embodiment, the insulation body may comprises as material a thermoplastic material, in particular polypropylene (PP) or polyethylene (PE), in particular may consist of this, preferably a plastic injection molded part, in order to provide electrical insulation. Plastics are good electrical insulators and may be produced easily and inexpensively. In addition, materials such as polypropylene (PP) and polyethylene (PE) are biocompatible.

In particular, the insulation body may be configured in at least two parts and may have a main body with a receptacle, in particular a recess, for receiving the transponder, and furthermore may have a closure body, in particular a type of complementary lid or plug, which is connectable to the main body via a form-fit and/or force-fit and hermetically seals the receptacle from an environment, so that the transponder is fixed in position and housed in a loss-proof manner. The at least two-part configuration permits simple manufacture of the two bodies, insertion of the transponder into the (transponder) receptacle and simple closure of this receptacle with the transponder inserted in order to keep it loss-proof and also to ensure sterility of the usually non-sterile transponder with respect to the environment.

In particular, the insulation body may be configured to be sterile or sterilizable.

Preferably, the closure body may be firmly bonded to the main body via hot deformation or ultrasonic welding. In this way, the closure body cannot accidentally fall off the main body and a hermetic seal of the transponder is also ensured.

According to one embodiment, the insulation body may have two through channels/passage openings, in particular slits, which run symmetrically to a longitudinal axis or plane of symmetry of the insulation body, and in particular run parallel, into which the first electrode and the second electrode are respectively insertable or inserted. The electrodes may respectively protrude/project/stick out distally and proximally from the insulation body and may in particular be held in the insulation body by a press fit. This configuration of the insulation body means that it is configured as a connecting body for the two electrodes. These electrodes are inserted into the passage openings and protrude both distally in order to be able to manipulate a patient's tissue and also protrude proximally from the insulation body in order to be connected electrically accordingly. Such a configuration with the two passage openings, in particular slits, is an efficient and cost-effective solution.

According to a further embodiment, the insulation body, in particular the slit, may have a tapering or a latch projection in the passage opening, in particular a latch projection configured complementary to the screen opening, so that the associated electrode is elastically held/fixed with the screen opening via an elastic form-fit via the latch projection (as tapering). If the screen opening is integrated in the electrode and thus a recess or hole is present, so to speak, and the electrode is seated (after insertion) in the passage opening, wherein the screen opening is in the region of the transponder, the geometry of the screen opening may be used to achieve an elastic (and thus also releasable) mount together with a complementary latch projection, which can, however, only be released with a predefined amount of force. The latch projection protrudes into the screen opening and is elastically preloaded. In particular, a ramp structure may be used as a latch structure to simplify assembly but may make disassembly more difficult. The electrode can thus be simply and easily pushed into the passage opening of the insulation body, wherein the electrode slides in over the ramp, and as soon as the ramp-shaped latch projection protrudes into the screen opening, it forms an undercut, such as a vertical undercut, so that disassembly can only be carried out by dissolving the undercut, for example by elastically pulling the latch projection back out of the screen opening.

In particular, the insulation body, in particular the insulation body and the first and second electrodes, may be configured symmetrically to a plane of symmetry, so that the transponder is also arranged symmetrically between the first and second electrodes.

Further preferably, the screen opening which serves as a passage for electromagnetic waves or signals, in particular radio signals, is configured to be elongated or slit-shaped and has a (slit) width which corresponds to the product of the coil diameter (of the transponder, in particular of the glass tag) and an ideal factor, wherein the ideal factor is in the region of 1.3 to 2.2, further preferably in a region of 1.6 to 1.9 and particularly preferably is 1.75. In other words, the screen opening, which is parallel to the transponder, in particular to the glass tag, has a width which is greater than the coil diameter and/or a width of the transponder, in particular a diameter of the glass tag.

Further preferably, the elongated or slit-shaped screen opening has a length which is −30% up to +50% of the total length of a coil core, in particular ferrite core, of the transponder, further preferably a length of 0% up to +30% of the total length of the ferrite core and particularly preferably a length of +15% of the total length of the ferrite core. In other words, the screen opening, which is parallel to the transponder, in particular to the glass tag, has a length which is preferably greater than the length of the coil core, in particular the ferrite core, and/or the transponder, in particular the glass tag.

Preferably, the insulation body, in particular the upper side, may have a color or be color-coded, so that information is assigned to the insulation body and thus to the electrode device or the medical instrument via color coding. In this way, an instrument system with a matching set of medical instrument and compatible transponder may also be created in particular.

According to one embodiment, the transponder may have a cylindrical shape, in particular with rounded ends, with a longitudinal axis of the transponder, the screen opening of the screen may be configured to be elongated or slit-shaped or slot-shaped and may have a longitudinal axis of the screen opening, and the longitudinal axis of the transponder may be parallel to the longitudinal axis of the screen opening, spaced apart from it and in particular arranged symmetrically to it. This means that the transponder is arranged symmetrically to the screen openings of the electrodes and can be read very easily.

Preferably, a distance between the transponder and the screen opening may be a minimum of 2 mm and/or a maximum of 20 mm, in particular between a longitudinal axis of the transponder and a longitudinal axis of the screen opening; and/or a shortest distance between the first electrode and the second electrode may be a minimum of 4 mm and/or a maximum of 40 mm.

In particular, the electrode is configured to be flat or sheet-like, in particular flat/planar, and has (in the region of the screen opening) a constant height (thickness) perpendicular to the screen opening.

In particular, the electrode may be made entirely of metal. Preferably, the electrode is made of stainless steel. Metal is impermeable to electromagnetic waves. Stainless steel is particularly easy to sterilize.

In particular, a cylindrical receptacle in the form of a recess may be configured in the insulation body, which has a proximal or distal opening in order to insert the transponder from the proximal or distal side.

Furthermore, in particular the transponder may be arranged at a distance from the screen opening and may be set back.

In particular, the receptacle/recess in the insulation body may be precisely matched to a glass tag as a transponder so that it does not fall out and is held in place by friction. Preferably, a press fit of the transponder may be configured in the receptacle of the insulation body, i.e. a smaller diameter of the receptacle than the diameter of a glass tag. In particular, the transponder may be housed in the receptacle of the insulation body via a press fit. Transponder and insulation body are therefore matched to each other in such a way that the transponder forms a press fit in the inserted state.

In particular, the electrode, in particular the first and second electrodes, may also form a press fit with the insulation body. The electrode and insulation body are therefore matched to each other in such a way that the electrode forms a press fit when inserted.

In particular, the transponder may be assembled in the insulation body, and in particular also the electrodes, without tools and/or may be inserted into and removed from the prepared receptacle (or passage opening) without tools. The electrode device can therefore be assembled without tools. In particular, the receptacle in the insulation body may be configured as a cylindrical recess or bore.

In particular, the width of the screen opening (i.e. perpendicular to a longitudinal axis of the electrode) may be a minimum of 5 mm and/or a maximum of 15 mm.

In particular, a distance (from the receptacle of the transponder and thus) of the transponder to the passage opening in which the electrode is inserted may be a minimum of 2 mm and/or a maximum of 20 mm.

In particular, the insulation body may be configured or manufactured as an off-tools plastic injection molded part. In particular, the insulation body is based on the idea that if in particular a signal-permeable insulation body is chosen (comprising e.g. thermoplastics, thermosets, plastics in general and/or silicone as material), the read and/or write distance is optimized by an electrode at least partially surrounding the transponder as metal shield/reflector/screen with screen opening, which distances the transponder from the upper side and thus from a read and write device which may be arranged there, with a geometrically defined opening.

According to a further embodiment of the invention, the insulation body and/or the electrode in the region of the screen opening may be provided with a biocide, so that the risk of germ formation is further reduced.

Preferably, the transponder may be a passive RFID transponder.

Preferably, the transponder is an RFID transponder, particularly preferably a glass tag, for storing information associated with a specific medical instrument. This RFID transponder is adapted to be individualized according to the requirements of a predefined process. In particular, the RFID transponder or RFID tag has:
a microchip, preferably with a dimension of less than 2 millimeters,
an antenna, preferably in the form of a coil, particularly preferably with an internal rod-shaped ferrite core around which the coil is wound, and
a sheathing, wherein the sheathing is preferably waterproof and/or airtight and preferably protects the electronics of the transponder from the environment.

According to a further embodiment, the transponder may also be an active RFID transponder that has at least one energy source, preferably in the form of a battery, an accumulator and/or a capacitor.

Preferably, the transponder is provided and adapted to store at least one of the following information in encrypted or unencrypted form:
general condition,
service life/end of service life,
maintenance interval,
performance and suitability for follow-up surgery,
reduced maintenance of product and possible product damage,
temperature overshoots and undershoots and possible product damage,
item number,
serial number, and/or
customer.

This makes it possible to detect and to count processing cycles for medical instruments, in particular in combination with individualized storage, and to save this information in the medical instrument itself. In particular, it may be detected whether all necessary process steps have been observed and carried out. A number of processing cycles may in particular be a proportional measure of the above information.

Preferably, the transponder has a cylindrical shape with rounded ends. The shape of the transponder, in particular the glass tag, is in particular a pill shape.

The insulation body with the receptacle for the transponder may in particular either remain free/open to the outside or may be filled/closed with a material permeable to signals. In particular, the transponder may already be shaped per se in the manner of a sealing cap in such a way that when the transponder is inserted into the insulation body, this or its opening is sealed (water/air-tight) to the outside by the transponder itself.

Preferably, the transponder is arranged centrally and symmetrically to each other in relation to the screen opening when viewed from the surroundings in the direction of the two electrodes as an extension. As a result, the transponder is centered in the screen opening and reception is improved.

According to a preferred embodiment, the size of a glass tag may be 2 mm in diameter and 12 mm in length. Alternatively, the dimension may preferably also be 3 mm in diameter and 13 mm in length or preferably 4 mm in diameter and 22 mm in length.

In addition, the transponder may preferably use a frequency band in the range from 12 to 15 MHz, advantageously in the range from 13 to 14 MHz, more preferably in the range from 13.4 to 13.7 MHz and particularly preferably of 13.56 MHz.

The object of the present disclosure is solved with respect to a medical transponder system/transponder communication system according to the invention in that it comprises a medical instrument with transponder according to the disclosure and a reading and/or writing device which may be coupleable to the transponder via signal technology and which is configured in particular with or itself as an instrument holder which is adapted to hold or temporarily fix the medical instrument with the transponder in a predetermined position and/or alignment relative to the reading and/or writing device, in which a signal transmission between the transponder and the reading and/or writing device is enabled. In other words, the medical instrument or the medical device may be read and/or written by a reading and/or writing device which may be brought into close proximity, i.e. at a distance of less than one centimeter in particular, to the transponder. In the case of medical instruments which have a connection, for example for an air supply, a power supply and/or a data exchange, this reading device may be attached to the medical device in the couplable adapter for the counterpart.

The object of the present disclosure is solved with respect to a production method for an electrosurgical instrument, in particular for an instrument according to the present disclosure, by the steps of: producing, in particular off-tool plastic injection molding, an insulation body with two passage openings and a receptacle for a transponder; installing or providing a screen opening in a first and/or in a second electrode; inserting the first electrode into the first passage opening and the second electrode into the second passage opening, inserting the transponder into the receptacle, and preferably closing the receptacle to receive the transponder in a loss-proof manner, in particular hermetically sealing it.

According to one variant, closing of the receptacle may be performed by gluing or casting, or the insulation body may be closed with a main body with the receptacle via a separate closure body, and this closure body in particular may be firmly bonded to the main body via hot deformation or ultrasonic welding.

### BRIEF DESCRIPTION OF THE DRAWINGS

The present disclosure is explained below based on preferred embodiments with reference to the accompanying Figures. The following is shown:

FIG. 1 shows a perspective view of an electrosurgical HF instrument according to a first preferred embodiment of the present disclosure;

FIG. 2 shows a perspective longitudinal sectional view of an electrosurgical HF instrument according to a further, second preferred embodiment of the present disclosure;

FIGS. 3 and 4 show each a further perspective view of the instrument from FIG. 2;

FIG. 5 shows a front view of a longitudinal section of the instrument from FIGS. 2 to 4;

FIG. 6 shows a perspective and partially transparent view of an electrosurgical HF instrument according to a further, third preferred embodiment of the present disclosure, in which the receptacle is closed with a plug;

FIGS. 7 to 11 show different sectional views of an electrosurgical HF instrument according to a further, fourth preferred embodiment of the present disclosure, in which a main body is connected to a closure body;

FIGS. 12 to 16 show different sectional views of an electrosurgical HF instrument according to a further, fifth preferred embodiment of the present disclosure;

The Figures are schematic in nature and are intended only to aid understanding of the invention. Identical elements are marked with the same reference signs. The features of the various configuration examples may be interchanged.

### DETAILED DESCRIPTION

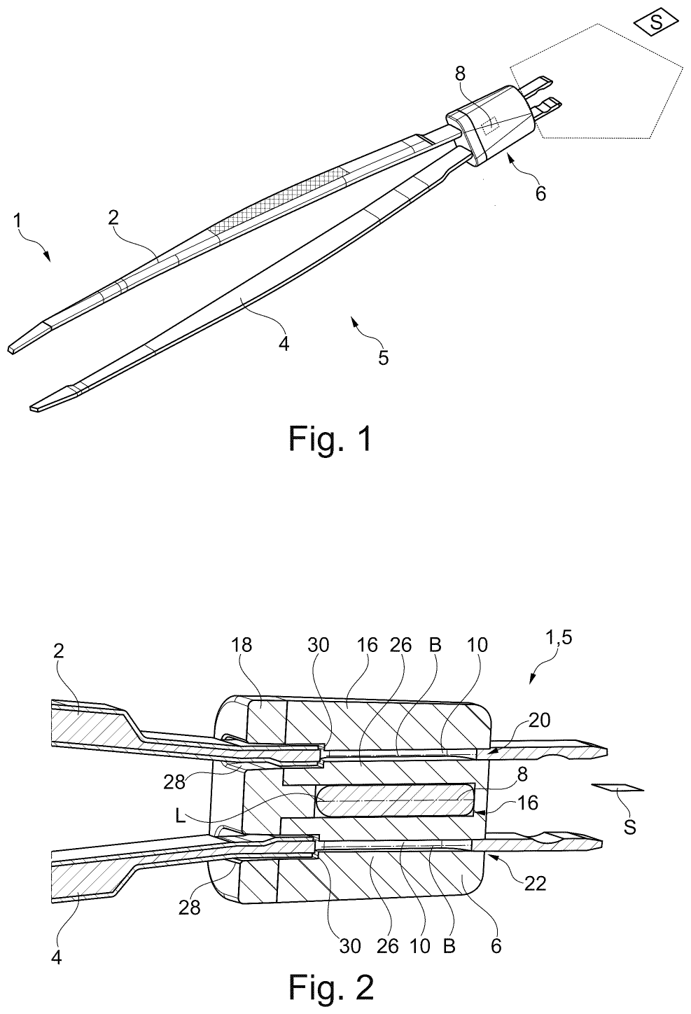

FIG. 1 shows an electrosurgical instrument 1 according to a first preferred embodiment of the present disclosure.

The electrosurgical instrument 1 (hereinafter referred to only as the instrument) is configured in the form of a bipolar HF instrument and has a first electrode 2 and a second electrode 4, which lie opposite each other distally of the instrument. Furthermore, the instrument 1 has an insulation body 6 in which a transponder 8 in the form of a glass transponder is housed. The insulation body 6 together with the electrodes 2, 4 and the transponder 8 form an electrode device 5 as an assembly for the instrument 1. This electrode device may be connectable to and disconnectable from the instrument.

The insulation body connects the first electrode 2 and the second electrode 4 with each other and thus forms a kind of geometric housing for the two electrodes 2, 4 and the transponder 8. Specifically, the transponder 8 housed in the insulation body 6 is arranged symmetrically and centrally between the first electrode 2 and the second electrode 4 from a structural or geometric point of view. It can also be said that the first electrode 2, the transponder 8 and the second electrode 4 are lined up in this order symmetrically to each other on a straight line transverse to a longitudinal axis of the insulation body 6.

In this particular embodiment, a screen opening 10 in the form of a slot hole is configured both in the first electrode 2 and in the second electrode 4 in the region of the transponder 8 or at the level of the transponder 8, so that the electrodes 2, 4 form a screen 12 impermeable to signals of electromagnetic waves with a defined screen opening 10 permeable to signals.

In this way, the transponder may be read 360°around the instrument 1 or the electrode device 5.

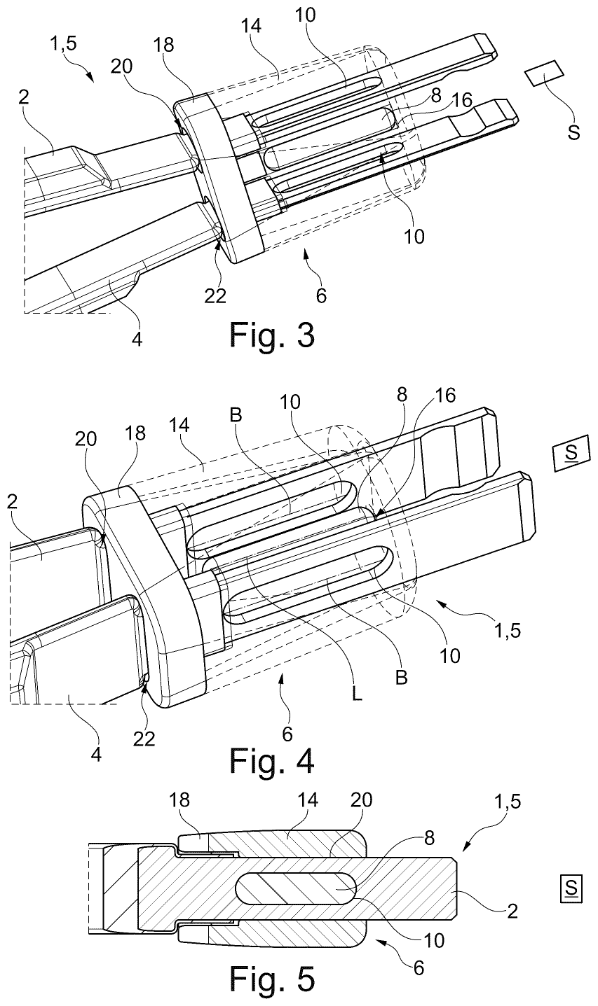

The HF instrument 1 therefore has a proximal insulation body 6 on its electrode device 5 (viewed on a longitudinal axis of the instrument), which is electrically insulating. In this embodiment, this insulation body 6 is made in one piece and has two passage openings 20, 22 in the form of slits 24 for receiving or inserting of the first and second electrodes 2, 4. Receiving and fixed connection of the electrodes 2, 4 to the insulation body 6 is made via a press fit (for a more detailed view of the individual features, reference is also made to FIGS. 2 to 5, which are also largely found in the embodiment of FIG. 1).

The insulation body 6 (i.e. also the electrode device 5) is symmetrical to a plane of symmetry S. In the region of the two opposing screen openings 10, which provide the transponder 8 centrally between them in extension, a latch projection 26 is configured complementary to the slot hole, which engages in a form-fitting manner in the associated screen opening 10. Since the insulation body 6 is manufactured as a (partially) elastic plastic (injection-molded part), the latch projection 26 may also deform (slightly) elastically perpendicular to a longitudinal axis of the electrode device or perpendicular to the plane of symmetry. The electrodes may therefore be inserted from distal to proximal into the passage openings in the assembled state (i.e. with the transponder 8 inserted and with the receptacle closed) of the insulation body and latch elastically when the latch projection 26 is inserted into the screen opening 10.

FIGS. 2 to 5 show different views of a further embodiment of an electrosurgical instrument 1 of the present disclosure.

The transponder 8 in the form of the glass transponder (glass tag as RFID transponder) is again located or arranged centrally in the proximal insulation body 6 in the recess provided for this purpose (which forms the receptacle 16). Due to this arrangement, the RFID (glass tag) transponder 8 is again located exactly between the electrodes 2, 4, which protrude proximally as connection contacts beyond the insulation body 6. The transponder 8 may be read in all directions or through 360° due to a corresponding geometric recess of the opposing electrodes in the region of the transponder 8 and within the proximal insulation body 6. The geometric configuration via the spacing of the electrodes 2, 4 as well as the possible definition of the screen openings 10 or recesses on the electrodes 2, 4 as elongated slots with a defined length and width may also influence the signal strength or the distance to the read and write device. Especially when using NFC technology as a transponder and reading/writing device, the very short read and write distance to the reading/writing device can be significantly increased.

In this embodiment of FIGS. 2 to 5, the insulation body 6 is configured in two parts to securely house the transponder 8 in the form of the glass tag. The insulation body 6 has a main body 14 with a receptacle recess (in this embodiment as a simple hole) as receptacle 16 and a second closure body 18 acting as a kind of complementary lid. Since the insulation body 6 in this embodiment is configured as an off-tools plastic injection molded part, the receptacle (recess) 16 for the transponder 8 can be provided directly.

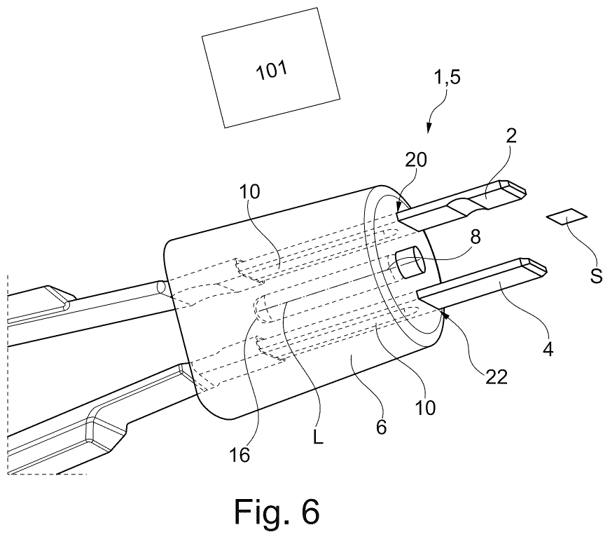

The main body 14 is coupled to the closure body 18 via both a form-fit and an elastic frictional connection (elastically configured insulation body 6). In addition, further passage openings 20, 22 are also configured in the closure body 18, which each comprise the electrodes 2, 4 and also realize a frictional connection via these (similar to a one-piece insulation body, as shown in FIG. 6 below). The closure body 18 (as a type of lid) and main body 14 may therefore be assembled via form-fit and/or force-fit, as is also the case with a one-piece insulation body 6 via the electrodes 2, 4.

The pill-shaped transponder 8 is again, when viewed in a direction perpendicular to the plane of symmetry S, aligned centrally between the two concentrically arranged screen openings 10, so that a good readout modality perpendicular to the plane of symmetry S is also provided. Specifically, the slot hole (screen opening 10) is configured with the same dimensions of length and width as the pill-shaped transponder 8. As a result, the transponder 8 is arranged symmetrically and in extension to the screen openings 10 as well as set back, and the electromagnetic radiation may be bundled with a corresponding improvement in a data connection.

In this embodiment, the closure body 18 is configured as a kind of plate with two slits 24 perpendicular to the plate, wherein the slits 24 have a funnel-shaped inlet 28 distally, i.e. in the direction from which the electrodes 2, 4 are inserted, in order to even further improve insertion. In the middle between the two parallel slits 24, the plate has a cylindrical base with the same or slightly larger diameter (press fit) as the cylindrical receptacle 16 of the main body 14. In this way, the closure body 18, when placed on the main body 18, is held in a force-fit manner (via friction or press fit) and the receptacle 16 is sealed off from the environment. In this way, the insulation body may be sterilized with the transponder 8, for example.

In this embodiment, in addition to the plane of symmetry S, there is even a second plane of symmetry (perpendicular to the plane of symmetry S) in the longitudinal section direction, as shown in FIG. 2.

The main body 14 also has a step 30 in the distal direction, as a kind of stop, so that the electrode 2, 4 can only be inserted up to this stop and, for geometric reasons, no further insertion is possible (the electrode cannot be inadvertently pushed excessively into the insulation body 6. This configuration with the stop 30 is also provided in the embodiment explained below.

FIG. 6 shows a further embodiment of an electrosurgical instrument 1, which essentially differs from the above embodiment only in that the insulation body 6 is made in one piece and is closed via an adhesive (in contrast to the two-part embodiment and not via a closure body). The insulation body 6 again has a recess in the middle as a receptacle 16 for the transponder 8, which is inserted from the proximal side. Finally, the receptacle 16 with the inserted transponder 8 is directly hermetically sealed (fluid-tight) via an adhesive or potting compound. Alternatively, a type of plug (as a closure body; not shown here) may also be used and this may be connected to the insulation body (as the main body) via hot deformation or ultrasonic welding.

Data of the transponder 8 may be read out and also written via a transponder communication system 101 of a preferred embodiment, shown schematically. The transponder communication system has a reading and/or writing device (not shown here) which may be coupled to the transponder 8 of the medical instrument 1 in terms of signal technology, which in particular is configured with or itself as an instrument holder, and which is adapted to hold or temporarily fix the medical instrument 1 with the transponder in a predetermined position and/or alignment relative to the reading and/or writing device, in which signal transmission between the transponder 8 and the reading and/or writing device is enabled.

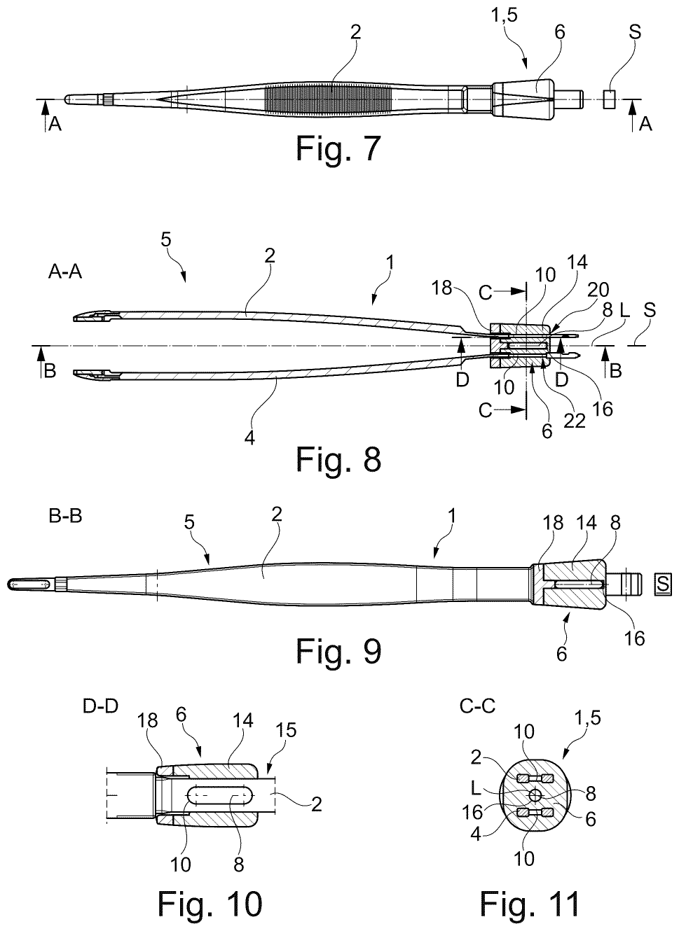

FIGS. 7 to 11 show different views of a further embodiment of the medical instrument 1, which has a two-part insulation body 6 in which the transponder 8 is housed.

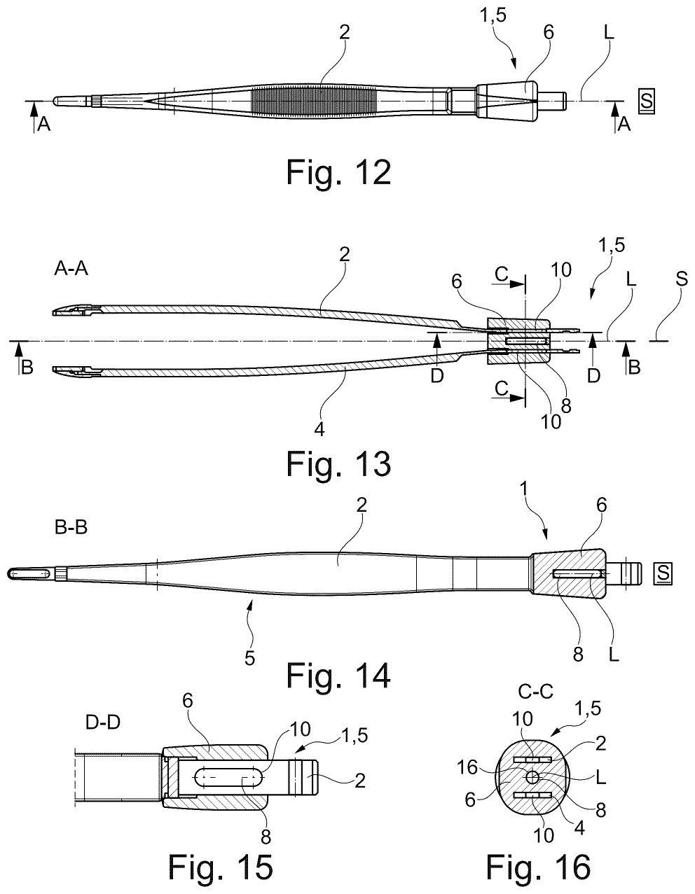

FIGS. 12 to 16 show a further embodiment of the electrosurgical instrument 1 according to the present disclosure. In this embodiment, the insulation body is again configured in one piece and is glued at the end face so that the transponder 8 is hermetically enclosed and sealed fluid-tight in the insulation body 6 with respect to the environment.

According to a preferred production method for an electrosurgical instrument according to the present disclosure, the instrument is manufactured and assembled via the following steps.

Providing S1, in particular producing and providing, by off-tool plastic injection molding of a two-part insulation body 6 with two passage openings 20, 22 for electrodes 2, 4 and a receptacle 16 for a transponder 8.

This is followed by the step of installing/creating or providing S2 a screen opening 10 in the first electrode 2 and the second electrode 4 of the instrument 1.

In the next step, the first electrode 2 is inserted S3 into the first passage opening 20 and the second electrode 4 into the second passage opening 22.

Finally, in step inserting S4, the transponder 8 is inserted into the receptacle 16, and the receptacle 16 is closed via an adhesive mass or via a closure body in order to receive the transponder 8 in a loss-proof manner, in order to fix it in its position, in particular in its position opposite the two screen openings 10 of the electrodes 2, 4, and to hermetically enclose it in order to ensure sterility.

### LIST OF REFERENCE SIGNS

1 electrosurgical instrument
2 first electrode
4 second electrode
5 electrode device
6 insulation body
8 transponder/glass tag
10 screen opening
12 screen
14 main body
16 receptacle
18 closure body
20 first passage opening
22 second passage opening
24 slit
26 latch projection
28 funnel-shaped inlet
30 step
101 transponder communication system
S plane of symmetry
L longitudinal axis
B longitudinal axis of the screen opening
S1 step producing insulation body
S2 installing screen opening in electrode
S3 inserting electrode in passage opening
S4 inserting transponder

1-14. (canceled)

15. An electrosurgical instrument comprising:
an electrode device having a first electrode and a second electrode positioned opposite to the first electrode;
an insulation body that connects the first electrode to the second electrode; and
a transponder that is housed in the insulation body,
the transponder being arranged between the first electrode and the second electrode, and
at least one screen opening located in the first electrode and/or the second electrode, the at least one screen opening located in proximity to the transponder so as to form a screen that is impermeable to signals of electromagnetic waves, with the at least one screen opening being permeable to signals of electromagnetic waves.

16. The electrosurgical instrument according to claim 15, wherein the at least one screen opening is elongate.

17. The electrosurgical instrument according to claim 15, wherein the at least one screen opening is slit-shaped or slot-shaped.

18. The electrosurgical instrument according to claim 15, wherein the insulation body comprises a thermoplastic material that provides electrical insulation.

19. The electrosurgical instrument according to claim 15, wherein:
the insulation body is configured in at least two parts comprising a main body and a closure body,
the main body defines a receptacle for receiving the transponder, and
the closure body is connectable to the main body via a form-fit and/or force-fit and hermetically seals the receptacle so that the transponder is housed in a loss-proof manner.

20. The electrosurgical instrument according to claim 19, wherein the closure body is firmly bonded to the main body via hot deformation or ultrasonic welding to provide a permanent fixation and hermetic seal.

21. The electrosurgical instrument according to claim 15, wherein:
the insulation body has a first passage opening and a second passage opening,
the first passage opening and the second passage opening run symmetrically to a longitudinal axis of the insulation body,
the first electrode is inserted into the first passage opening and the second electrode is inserted in the second passage opening, and
each of the first electrode and the second electrode protrudes proximally from and distally from the insulation body.

22. The electrosurgical instrument according to claim 21, wherein:
the at least one screen opening comprises a first screen opening in the first electrode and a second screen opening in the second electrode,
the first passage opening comprises a first tapering or a latch projection configured to hold the first electrode with the first screen opening in an elastic form-fit with the first tapering or a latch projection, and
the second passage opening comprises a second tapering or a latch projection configured to hold the second electrode with the second screen opening in an elastic form-fit with the second tapering or a latch projection.

23. The electrosurgical instrument according to claim 15, wherein the insulation body is configured symmetrically to a plane of symmetry, so that the transponder is also arranged symmetrically between the first electrode and the second electrode.

24. The electrosurgical instrument according to claim 15, wherein:
the transponder has a cylindrical shape with a first longitudinal axis,
the at least one screen opening has a second longitudinal axis, and
the first longitudinal axis is parallel to and spaced apart from the second longitudinal axis.

25. The electrosurgical instrument according to claim 15, wherein a distance between the transponder and the at least one screen opening is a minimum of 2 mm and/or a maximum of 20 mm.

26. The electrosurgical instrument according to claim 15, wherein a shortest distance between the first electrode and the second electrode is a minimum of 4 mm and/or a maximum of 40 mm.

27. The electrosurgical instrument according to claim 15, wherein the insulation body defines a cylindrical receptacle that forms a recess, the recess having an opening in a proximal and/or a distal direction to facilitate insertion of the transponder from a proximal side and/or distal side.

28. A medical transponder communication system comprising:
the electrosurgical instrument according to claim 15; and
a reading and/or writing device,
the reading and/or writing device being coupleable to the transponder of the electrosurgical instrument via signal technology, and
the reading and/or writing device adapted to hold or temporarily fix the electrosurgical instrument with the transponder in a predetermined position and/or alignment relative to the reading and/or writing device, in which a signal transmission between the transponder and the reading and/or writing device is enabled.

29. A method for producing an electrosurgical instrument comprising the steps of:
producing an insulation body defining a first passage opening for receiving a first electrode, a second passage opening for receiving a second electrode, and a receptacle for receiving a transponder;
forming or providing a screen opening in the first electrode and/or in the second electrode;
inserting the first electrode into the first passage opening;
inserting the second electrode into the second passage opening; and
inserting the transponder into the receptacle.

30. The method according to claim 29, further comprising the step of closing the receptacle so that the transponder is received in the receptacle in a loss-proof manner.

31. The method according to claim 30, wherein the step of closing the receptacle comprises gluing or casting.

32. The method according to claim 30, wherein the insulation body is closed with a main body with the receptacle via a separate closure body, the separate closure body being bonded to the main body via hot deformation or ultrasonic welding.
